(19) United States
(12) Patent Application Publication
(10) Pub. No.: US 2019/0355482 A1
(43) Pub. Date: Nov. 21, 2019

(54) SYSTEMS AND METHODS FOR PROVIDING A SUBSTANTIALLY UNIFIED MULTIFUNCTION VIRTUAL HEALTH AND WELLNESS PLATFORM

(71) Applicants: Sabina P Taneja, Wyckoff, NJ (US); Samir S Taneja, Wyckoff, NJ (US)

(72) Inventors: Sabina P Taneja, Wyckoff, NJ (US); Samir S Taneja, Wyckoff, NJ (US)

(21) Appl. No.: 16/412,504

(22) Filed: May 15, 2019

Related U.S. Application Data
(60) Provisional application No. 62/672,098, filed on May 16, 2018.

(51) Int. Cl. G16H 80/00; G06F 9/451; H04L 29/06; H04L 29/08

(52) U.S. Cl. CPC G16H 80/00; G06F 9/451; H04L 51/046; H04L 67/12; H04L 67/1097; H04L 67/42

(57) ABSTRACT

The present invention may include providing a substantially unified multifunction virtual health and wellness system and may include providing a server that includes a processor and a memory device. The system and method may further include providing a plurality of local and remote communications devices and control modules that are operably connected to a cloud-based application. Providing a client device operably connected to the server using a cloud-based server application using at least one of a plurality of communications devices and control modules. The cloud-based application processes data received from the client device using the server processor. The processed data is communicated bi-directionally between the server and the client device. The client device that includes a plurality of biometric sensors to measure a plurality of biometric readings the plurality of biometric readings may include a blood pressure reading, a temperature reading, a glucose reading, a cholesterol reading, a red blood cell count reading, and a white blood cell count reading. The client device may have an editable graphical representation of the human body allowing a user on the client device to graphically show points of issue on the editable graphical representation of the human body.

### RELATED APPLICATION

This application claims priority from U.S. provisional application Ser. No. 62/672,098 filed May 16, 2018, the contents of which are hereby incorporated by reference in its entirety.

### FIELD OF THE INVENTION

The present invention relates to online services. More particularly, the present invention relates to online and virtual health and wellness services provided to the elderly, disabled, immobile, and the infirm.

### BACKGROUND OF THE INVENTION

Telemedicine applications that exchange health information from one location to another are well known in the art. In general health care, telemedicine gives patients a sense that their illness is being monitored more closely, the ability to participate in their own health management, and a feeling they have not been forgotten by their doctor. Also well know are individual systems that provide entertainment, delivery of groceries and the like that make life for convenient for the user of these services.

However, the present invention ties telemedicine, entertainment, social mental health, pharmacies, grocery stores, transportation and the like into one multifunction virtual health and wellness platform that not only address the physical wellness of a patient but also the mental health and social health of patents to an online platform.

### SUMMARY OF THE INVENTION

Implementation of the present invention requires the design of a proprietary hardware and software to interface with existing virtual service that allow for the care of elderly and infirm clients. A feature of the present invention may be a dedicated computer terminal/interface to serve as a portal with the online service. The online communication may be activated and conducted with a touch screen, voice activation, visual activation or the like with a user interface/graphical user interface.

The present invention may further include online medical triage and referral services. This service may, for example, provide services such as scheduling of medical, dental, eye care and the like services. Other services may include one to one assessment with medical staff to determine the need for acute and chronic medical care. This aspect of the online community provides at least two fundamental services. First is to triage acute care in determining the severity of injuries, new symptoms, or medication side effects. The second is to provide management of chronic health care needs including medication schedule and compliance, prescription inventory and refill, follow-up of scheduled medical visits and hospitalizations, and assessing adequacy of response to existing therapies. In regard to the latter, the system would interface with a client's care team to ensure complete understanding of the individual medical needs and goals, and to communicate any concerns regarding efficacy and adherence.

The present invention may further include biometric sensors to monitor blood pressure, EKG, blood glucose, respiration, SPO2, temperature, or any other sensors to enable medical staff to triage the severity of acute problems and to monitor acute or chronic care.

The present invention may further include online social/gaming services—this is perhaps one of the most important aspects of the service as it would likely drive client interest the most. The purpose is to allow clients, who are limited in their physical capabilities, due to mobility limitations, to socialize within an online community of age and condition-matched peers who also subscribe to the service. Interaction may occur through moderated chat rooms, such as a book club, a movie with follow-up discussion, political discussions/current events, exercise classes, or games (bingo, trivia contests, online gaming and the like) which could be conducted in a group or one to one. One-on-one client based social interactions may be feasible as well but may need to be monitored (via key word, recording, and the like) for client security.

The present invention may further include mental health services such as counseling, group therapy, psychosocial assessments, and music therapy. For example, depression is a major problem among the elderly and the infirm, and online counseling, as offered in the present invention, may be a valuable tool to combat loneliness and depression among the elderly limited in their social support structure. Clients may access a social worker, a licensed therapist, or other mental health professionals on a regular or periodic basis to address acute or chronic mental health issues. Part of this service may be the ability to triage problem severity and determine the need to link clients with dedicated psychiatric services on an urgent or chronic basis.

Yet still another object of the present invention may include managing daily activities. For example, clients utilize the online to help organize daily services with third party vendors. This aspect of the present invention may include arrangements for online grocery shopping and delivery, delivery of necessary household items, managing finances, bill pay, organization of transportation, etc.

The present invention may maintain active partnerships with external vendors to provide the afore-mentioned services, which could be directly billed to clients or may be included in a part of the monthly costs of the present invention to clients.

The present invention may also include services to act as a liaison between clients and local healthcare, social, psychiatric services, as well as businesses providing services necessary for clients in managing their home and daily life. The individual needs of the client would be assessed, for example, upon enrollment to the agency to determine necessary social support. These services may be vetted by the service provider for reliability, pricing of services, accessibility, security and safety, and commitment to the elderly and infirm. If the present system did not include a specific service, referrals may be made to clients seeking specific services and may be added to the present invention offered services.

Yet another feature of the present invention may include security features for users of the platform. Exposure to the elderly and infirm to an online social group potentially exposes them to predators seeking to provide physical, financial, or emotional retribution/harm. As such, clients will be interviewed by the service provider prior to agreeing to enlist them in the service, both to allow assessment of their needs and their suitability for the service as it relates to the safety and privacy of other clients. Additionally, background checks may be conducted prior to acceptance as a client. The level of scrutiny of client communications needed to adequately protect the client will also be assessed on an individual basis.

An assessment of identity, background, and social situation would be necessary to determine suitability for the services. Online interactions may be recorded, monitored, scanned for key words, or the like to determine the potential for any abuse. A security measure that may be implemented may be that the computer hardware/software applications may be limited in functionality so as only to allow direct interaction with the online community without upload, recording, or introduction of other files, media, or websites from external sources.

According to an embodiment of the present invention a system for providing a substantially unified multifunction virtual health and wellness system may include a server that includes a processor and a memory device, a plurality of local and remote communications devices and control modules that are operably connected to a cloud-based application; a client device operably connected to the server using a cloud-based server application using at least one of a plurality of communications devices and control modules. The cloud-based application may process data received from the client device using the server processor. The processed data may be communicated bi-directionally between the server and the client device. The client device may include a plurality of biometric sensors to measure a plurality of biometric readings. The plurality of biometric readings may include a blood pressure reading, a temperature reading, a glucose reading, a cholesterol reading, a red blood cell count reading, and a white blood cell count reading. The client device may include an editable graphical representation of the human body allowing a user on the client device to graphically show points of issue on the editable graphical representation of the human body.

According to an embodiment of the present invention a method for providing a substantially unified multifunction virtual health and wellness system may include providing a server that includes a processor and a memory device. The method may further include providing a plurality of local and remote communications devices and control modules that are operably connected to a cloud-based application. Providing a client device operably connected to the server using a cloud-based server application using at least one of a plurality of communications devices and control modules. The cloud-based application processes data received from the client device using the server processor. The processed data is communicated bi-directionally between the server and the client device. The client device that includes a plurality of biometric sensors to measure a plurality of biometric readings the plurality of biometric readings may include a blood pressure reading, a temperature reading, a glucose reading, a cholesterol reading, a red blood cell count reading, and a white blood cell count reading. The client device may have an editable graphical representation of the human body allowing a user on the client device to graphically show points of issue on the editable graphical representation of the human body.

### BRIEF DESCRIPTION OF THE DRAWINGS

FIG. 1 is an illustration for a generalized graphical user interface in accordance with one embodiment of the present invention;

FIG. 2 is a block diagram in accordance with one possible embodiment of the present invention;

FIG. 3 is a schematic diagram of circuitry constructed in accordance with one possible embodiment of the present invention;

FIG. 4 is a block diagram in accordance with one possible embodiment of the present invention;

FIG. 5 is a block diagram in accordance with one possible embodiment of the present invention;

FIG. 6 is a generalized graphical representation of one embodiment of a human representation in accordance with one embodiment of the present invention;

FIG. 7 show a flow chart illustrating some of the steps involved in accordance with one embodiment of the present invention;

### DETAILED DESCRIPTION OF THE INVENTION

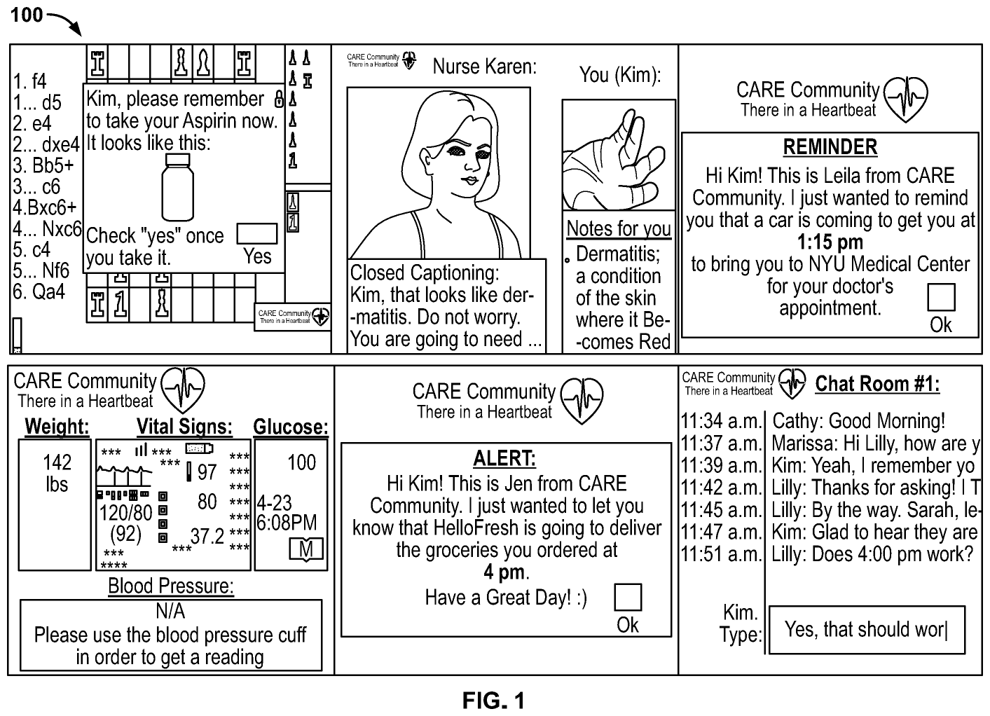

FIG. 1 shows a graphical user interface 100 for systems and methods of the present invention. As FIG. 1 shows, reminders, alerts, entertainment, social media, telemedicine, physical fitness, biometric data, etc. may be displayed to the user. The graphical user interface may be a touch screen, voice command activated, or may be used by any suitable means for communicating information to a user.

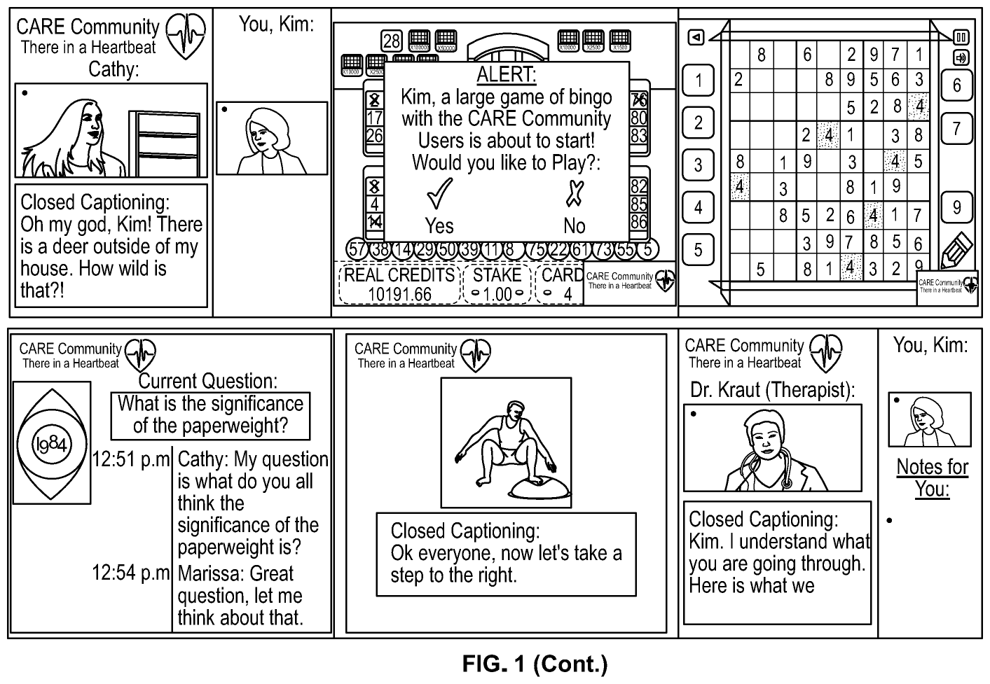

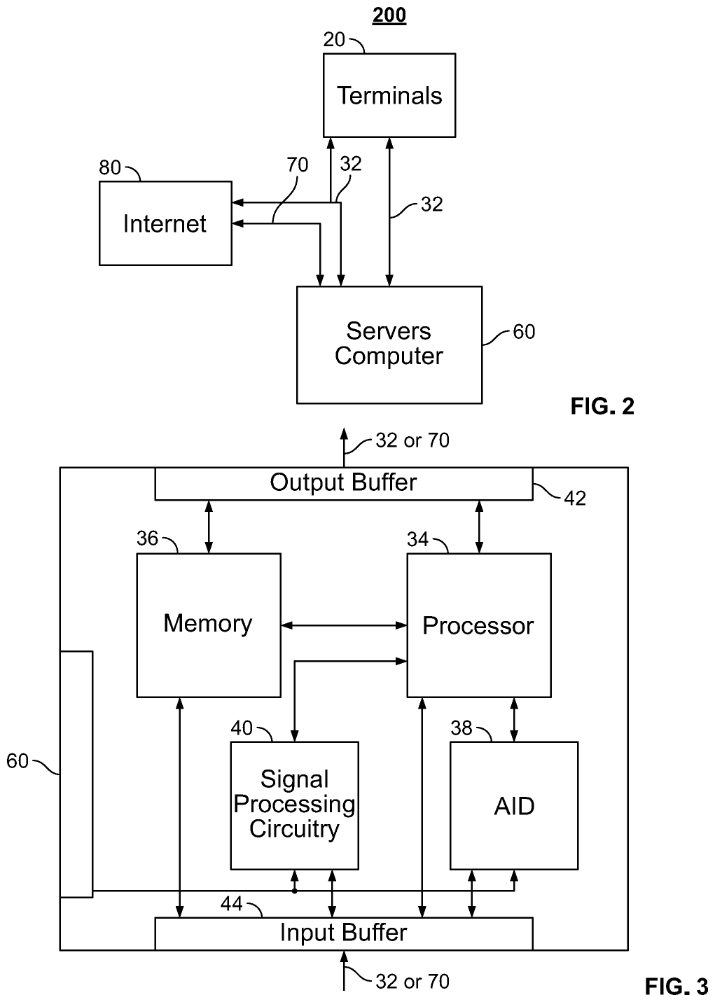

FIG. 2 shows an illustrative block diagram of a System 200 for system and methods of the present invention that may include one or more local or remotely connected servers and/or computers 60, and Terminals 20. Terminals 20 may include Patient Care Terminals (PCT). These terminals may be connected to the one or more Servers 60 and computers via any wireless or wired LAN system(s), telephone communication networks, satellite networks, cable television networks, TCP/IP to the Internet and/or any other suitable communications network or protocol.

Communications link 32 and 70 may preferably be a wireless communication link such as a cellular telephone link, a wireless Internet link, wireless network link, or a wireless modem for receiving information from a cloud-based application and one or more third-party servers (not shown).

Communications links 32, and 70 may be a hardwired link for use when wireless communication is not available or desired. Communications links 32, and 70 may be any suitable communications link such as a serial port, a parallel port, a universal serial bus (USB), an RS232, or GPIB link, a digital or analog modem, or a network interface link (e.g., Ethernet links, token ring links, etc.).

FIG. 3 shows a schematic diagram of circuitry constructed in accordance with one possible embodiment of a remote server according to the present invention. FIG. 3 includes a communications links 32 and 70, an input buffer 44, signal processing circuitry 40, and an A/D converter 38, a memory device 36, a processor 34 and an output buffer 42. Server 60 may be directly liked to an online cloud-based application such as medical, dental, mental health, entertainment, delivery services, transportation services and other desired services to enable a patient to have the feeling of independence while being under supervised care.

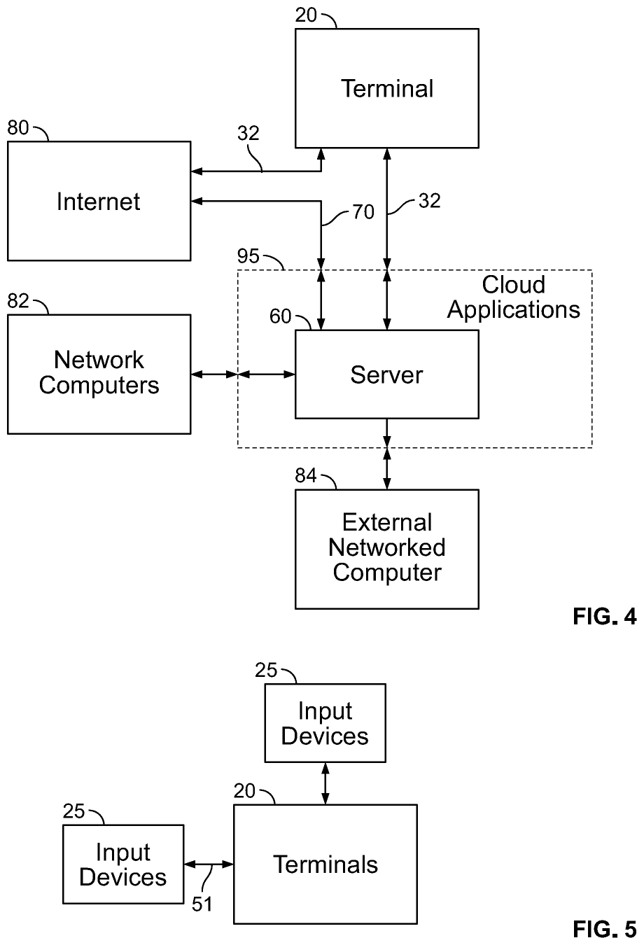

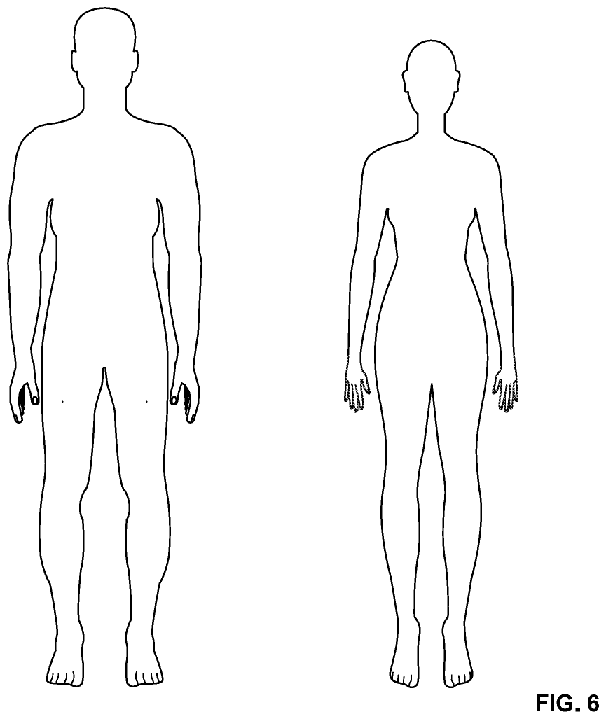

FIG. 4 shows one possible configuration of the present invention. FIG. 4 shows Terminal 20, Server 60, Cloud Applications 95, Internet 80, Networked Computers 82, External Networks 84 and Communications Links 32 and 70. Operating in conjunction with FIG. 4, may also be FIG. 5 that shows input devices 25 operably connected to Terminals 20. One example of operation of the present invention in operation may include Input Devices 25 that may include biometric sensors such as blood pressure sensors, temperature sensors, SPO2 sensors, A1C sensors or any other suitable sensor may be input to Terminals 20 via, for example, Link 51 which may be a wireless communication link such as a cellular telephone link, a wireless Internet link, wireless network link, or a wireless modem for sending information from biometric sensor information to such Terminal 20. For example, Link 51 may also be any suitable local, short, or intermediate range communications link such as a Bluetooth.TM. or IrDA infrared link. Any other type of appropriate wireless link may also be used if desired.

In operation, Input Device 25 may send biometric sensor information to Terminal 20. Such information may be the sent to Cloud-Based Application 95 via any know protocol and such information may then be sent to Server 60. Other Networked Computers 82 may be used to read such information and send information back to Terminal 20 or other External Networked Computer 84. For example, a patent may not be feeling well and have a temperature of 101, this information is read by input device 25 and transmitted to Terminal 20. The information is then sent to Cloud-Based Application 95 and further sent to Server 60. The temperature information may be stored in Server Memory device 36 (shown in FIG. 3) and disseminated to Networked Computer 82 or External Networked Computer 84. Of course, other information on the patient may be necessary and such information may be obtained by other sensors as well as other patient information stored within Server 60. The information that is obtained may allow a diagnosis and treatment plan that may be sent to direct care providers who monitor and may administer treatment to the patient.

FIG. 6. Shows a graphical user interface display of a human body that allows the user to mark and transmit information to server 60. As part of the virtual health and wellness system a graphical user interface display of a human body may be provided on Terminal 20 or cloud-based application 95 to allow the user to mark and transmit information to server 60 via a cloud-based application 95. The graphical user interface display of a human body may use touch screen technology, or any other suitable technology to mark areas of concern for the patient. This information may be used in conjunction with other biometric information received from Input devices 25, stored patient information from memory device 36 on Server 60 to diagnose and treat the patient.

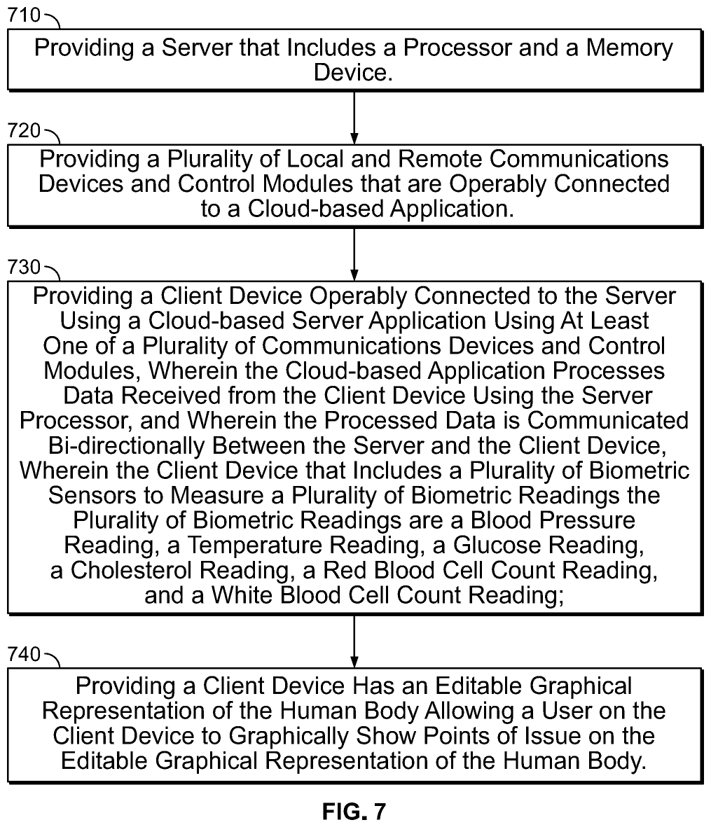

FIG. 7. shows an illustrative method according to an embodiment of the present invention that comprises the steps of providing a server that includes a processor and a memory device in step 710. In step 720, providing a plurality of local and remote communications devices and control modules that are operably connected to a cloud-based application. In step 730, providing a client device operably connected to the server using a cloud-based server application using at least one of a plurality of communications devices and control modules, wherein the cloud-based application processes data received from the client device using the server processor, and wherein the processed data is communicated bi-directionally between the server and the client device, wherein the client device that includes a plurality of biometric sensors to measure a plurality of biometric readings the plurality of biometric readings are a blood pressure reading, a temperature reading, a glucose reading, a cholesterol reading, a red blood cell count reading, and a white blood cell count reading. In step 740, Providing a client device has an editable graphical representation of the human body allowing a user on the client device to graphically show points of issue on the editable graphical representation of the human body.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

The reference numbers recited in the in this application are solely for ease of examination of this patent application, and are exemplary, and are not intended in any way to limit the scope of the claims to the particular features having the corresponding reference numbers in the drawings.

We claim:

1. A system for providing a substantially unified multifunction virtual health and wellness system comprising:
a server that includes a processor and a memory device,
a plurality of local and remote communications devices and control modules that are operably connected to a cloud-based application;
a client device operably connected to the server using a cloud-based server application using at least one of a plurality of communications devices and control modules, wherein the cloud-based application processes data received from the client device using the server processor, and wherein the processed data is communicated bi-directionally between the server and the client device.
wherein the client device that includes a plurality of biometric sensors to measure a plurality of biometric readings the plurality of biometric readings are a blood pressure reading, a temperature reading, a glucose reading, a cholesterol reading, a red blood cell count reading, and a white blood cell count reading; and
wherein the client device has an editable graphical representation of the human body allowing a user on the client device to graphically show points of issue on the editable graphical representation of the human body.

2. The system of claim 1, wherein the cloud-based application includes an application for physical healthcare, an application for mental healthcare, an application for fitness, an application for transportation, an application for grocery ordering and delivery, an application for pharmacy ordering and delivery, an application for pier to pier communication, an application for client-server communication, an application for entertainment, and a calendar application.

3. The system of claim 1, a user interface is installed on the one or more client devices.

4. The system of claim 1, wherein the cloud-based server application determines functional request dependencies from communication requests input by a user in the user interface when selecting messaging functions.

5. The system of claim 1, wherein cloud-based communications and control modules include a plurality of commerce-based applications.

6. The system of claim 5, wherein cloud-based communications and control modules include one or more entertainment applications.

7. The system of claim 5, wherein cloud-based communications and control modules include one or more document creation and management applications.

8. The system of claim 5, wherein cloud-based communications and control modules include one or more dashboard applications.

9. The system of claim 5, wherein cloud-based communications and control modules include one or more document creation and management applications.

10. The system of claim 5, wherein cloud-based communications and control modules includes one or more video call and multimedia messaging applications.

11. A method for providing a substantially unified multifunction virtual health and wellness system comprising the steps of:
providing a server that includes a processor and a memory device,
providing a plurality of local and remote communications devices and control modules that are operably connected to a cloud-based application;
providing a client device operably connected to the server using a cloud-based server application using at least one of a plurality of communications devices and control modules, wherein the cloud-based application processes data received from the client device using the server processor, and wherein the processed data is communicated bi-directionally between the server and the client device.
wherein the client device that includes a plurality of biometric sensors to measure a plurality of biometric readings the plurality of biometric readings are a blood pressure reading, a temperature reading, a glucose reading, a cholesterol reading, a red blood cell count reading, and a white blood cell count reading; and
wherein the client device has an editable graphical representation of the human body allowing a user on the client device to graphically show points of issue on the editable graphical representation of the human body.

12. The method of claim 11, wherein the cloud-based application includes an providing an application for physical healthcare, an application for mental healthcare, an application for fitness, an application for transportation, an application for grocery ordering and delivery, an application for pharmacy ordering and delivery, an application for pier to pier communication, an application for client-server communication, an application for entertainment, and a calendar application.

13. The method of claim 11, providing a user interface is installed on the one or more client devices.

14. The method of claim 11, wherein the cloud-based server application determines functional request dependencies from communication requests input by a user in the user interface when selecting messaging functions.

15. The method of claim 1, wherein cloud-based communications and control modules includes providing a plurality of commerce-based applications.

16. The method of claim 15, wherein cloud-based communications and control modules includes providing one or more entertainment applications.

17. The method of claim 15, wherein cloud-based communications and control modules includes providing one or more document creation and management applications.

18. The method of claim 15, wherein cloud-based communications and control modules includes providing one or more dashboard applications.

19. The method of claim 15, wherein cloud-based communications and control modules includes providing one or more document creation and management applications.

20. The method of claim 15, wherein cloud-based communications and control modules includes providing one or more video call and multimedia messaging applications.
